(19) United States
(12) Patent Application Publication
(10) Pub. No.: US 2024/0378388 A1
(43) Pub. Date: Nov. 14, 2024

(54) DATA EXTRACTION METHOD AND APPARATUS

(71) Applicant: Juniper Health, Inc., Orono, MN (US)

(72) Inventors: Sean C. Benson, Orono, MN (US); Dave Johnson, Orono, MN (US)

(21) Appl. No.: 18/658,259

(22) Filed: May 8, 2024

Related U.S. Application Data
(60) Provisional application No. 63/501,053, filed on May 9, 2023.

(51) Int. Cl. G06F 40/30; G06V 30/10; G16H 10/60

(52) U.S. Cl. CPC G06F 40/30; G06V 30/10; G16H 10/60

(57) ABSTRACT

The present invention relates to a method and apparatus for monitoring a stream of health care data, applying a rules-based methodology to process the data to determine how the data should be structured for abstraction to a data registry, and providing recommendations and rationales in support of the abstraction.

### RELATED APPLICATIONS

The present application claims priority to and incorporates by reference U.S. Patent Application No. 63/501,053 filed May 9, 2024.

### BACKGROUND OF THE INVENTION

### Field of the Invention

The present invention relates to a method and apparatus for monitoring a stream of health care data, applying a rules based methodology to process the data to determine how the data should be structured for abstraction to a data registry, and providing recommendations and rationales in support of the abstraction.

### Background

The health care services industry is a data intensive environment, where data collection and sharing are necessary and required at many different levels. This includes sharing data with various entities tasked with broad scale collection and aggregation of data for the purpose of analyzing, monitoring, and improving health care services, such as using data to improve quality of care, efficiency of care, costs based analysis, medical research, and improving patient outcomes.

One such area of data collection and analysis is the domain of various data registries, data banks, and data aggregation services. For example, the National Trauma Data Bank aggregates data from trauma centers for the purposes stated above. Many other similar organizations exist in other areas of health care, which provide similar services.

Accordingly, health care providers and systems must submit patient data to these centralized repositories. In some cases, submitting the data is legally required, in other cases it is a quasi-requirement, as data submission is necessary to obtain certification levels, to meet audit requirements, or for other reasons essential to providing health care services. Currently, health systems accomplish this by employing specialized registrars (or abstractors), who are individuals that abstract the clinical chart from the electronic health record software program (EHR), and manually prepare the relevant data for inclusion in the registry submission software.

This is a very complex and time-consuming process. The process has to be completed for every patient, and in some case multiple times for a patient, and requires a detailed knowledge of the health care services provided, the EHR software, and the registry submission software. Also, the registrars need to have a high degree of familiarity with health care practices to interpret the various records, and apply professional judgement to understand and translate the data between the various systems in an accurate, consistent, and efficient manner. In larger health care systems, this function requires multiple staff dedicated to the task full time.

In addition to the time data registry fulfillment takes, the process is also highly subjective as a great deal of interpretation and judgment is required to determine how EHR data should be treated in the registry. Thus, the process can vary significantly from person to person, and among different organizations. This subjectivity undermines the integrity of the data, and the conclusions drawn therefrom.

Thus, a need exists for a system and method for processing health records for compliance with data registry fulfillment that does not suffer from the drawbacks associated with the prior art.

### BRIEF DESCRIPTION OF THE FIGURES

FIG. 1 is a screen shot of a Pre-Abstract Patient List screen

FIG. 2 is a screen shot of a Patient Review screen.

FIG. 3 is a screen shot of an Abstract Patient List screen.

FIG. 4 is a screen shot of an Abstract Detail screen.

FIG. 5 is a screen shot of a Hospital Procedures functionality.

FIG. 6 is a screen shot of an Injury Diagnosis screen.

FIG. 7 is a block diagram of the software subsystems of the present invention.

FIG. 8 is a block diagram of the system architecture of the present invention.

FIG. 9 is an interface block diagram.

### DETAILED DESCRIPTION OF THE PREFERRED EMBODIMENTS

In the Figures, a system and method for automated processing of health care information for integration with a data registry requirements.

In general, the system comprises the following features, functions, and attributes (described at a high level of abstraction). First, patient specific health care information is obtained from a number of existing health systems and process, including EHRs, health systems such as emergency medical systems (EMS), billing systems, and repositories of scanned medical documents. A natural language processing (NLP) tool is used to interpret and structure all relevant data. The system then applies a series of rules based tasks and functions to the aforementioned structured patient data to automate and standardize the abstraction of specific data elements required for submission to clinical registries. The system then displays the relevant data fields, along with recommendations for how the data should be abstracted to the registry and provided the rationale for the recommendations. The rationale also includes hyperlinks to the relevant information imported from source systems.

The system that performs the functionality described herein generally is comprised of one or more general-purpose computers or computer systems functioning in a network environment. The system can operate in a SaaS or cloud environment where users access the system through a conventional browser. The functions are carried out through software applications executing on the foregoing computers, which can include desktops, laptops, tablets, servers, and the various network connections there between. In one embodiment, the system is a web/internet based application that requires an internet connection, and runs on servers operated by a commercial based cloud services provider.

In order to perform as described herein, the system requires certain integrations with other systems, and additional functionalities as follows. The system must acquire patient lists such as a list of patients who enter the ED/hospital, regularly updated. Preferably, the system requires a daily list of new patients. This list can be provided in various formats established for health care data (ex. HL7, CSV, FHIR, etc. . . . ). The system must be able to integrate with the various data systems (most notably EHR systems) described herein either through specialized integrations, APIs, data exchange tools, or through standardized industry data exchange protocols such the Fast Healthcare Interoperability Resources (FHIR). FHIR can be used to interface with clinical data in the EHRs. This data includes clinical documents, labs, vitals, and medications. Preferably, each day, the system analyzes the Patient List, and then accesses the clinical data via FHIR. The system should have access to billing data, which the system can access via different formats (ex. HL7 or UB-04 billing file). This information may not be immediately available, and is typically available a few days after discharge. The system should have access to emergency medical services (EMS) data, for example the system accesses source systems like ImageTrend's Health Information Hub product to access EMS data. The system should have access to an NLP interface where clinical documents are sent to the NLP service as they are received. The NLP service processes the documents and returns relevant medical phrases and associated codes, and provides further context based analysis of the document text. The system preferably utilizes an optical character recognition (OCR) service/technology. Scanned documents are analyzed by an OCR service. Examples of scanned documents are clinical documents from external health systems. The system accesses these scanned documents, then sends them to the OCR service. The service processes the documents and returns a text file, which is then sent to the NLP service for processing. Within the OCR software, configuration templates can be created to ensure that the OCR service can correctly analyze each type of scanned document. The system should have data exchange access to the applicable registries. Periodically, the system sends the finalized patient data to various centralized registries. These include the National Trauma Data Bank (NTDB) and state-specific trauma registries, and other registries.

The following describes in more substantial detail the mechanics of the system, and the processing steps carried out to accomplish the purpose of the present invention, beginning with the Pre-Abstraction process, which allows the registrars to review a list of new patients presented to the registrars by the system to determine how the patient's data should be abstracted to the registry. The system uses a rules engine to automatically provide a recommendation for whether the patient's data (or portions thereof) should be included in the registry. The registrar can review the system's recommendations and make a final determination.

The Pre-Abstract Patient List (shown in FIG. 1) is displayed upon logging into the system. The Pre-Abstract Patient List contains the following fields: Actions, Last Name, First Name, medical record number (MRN), Status, Date of Discharge, international classification of disease code (ICD-10), Description, and Recommendation. A Pre-Abstract/Abstract tab can be selected to move between the two screens (the Abstract screen described below).

In addition, each patient is assigned a Status that can be reviewed and confirmed by the registrar, such as those described below.

The system also provides a search field. The search field can be used to search for any patient in the system. Searches can be done on Last Name, First Name, and MRN.

The Column headers can be clicked on to sort the data in the grid.

The ICD-10 and Recommendation columns may contain more text than what can be presented in the grid. When that occurs, the system displays an ellipsis in those cells and the registrar can hover the cursor over the relevant cell to display the entire text in a balloon.

The purpose of the Pre-Abstraction display is to allow the registrar to review each patient in the list and assign a Status, which determines which patient data will be abstracted to the registry. The registrar can perform the following actions. Choose “Include” from the Action column: this changes the patient's Status to “Inclusion Confirmed”. The patient record is removed from this list shown above, and added to the Abstract Patient List. When this is chosen, an “Undo” button appears on the form that allows the registrar to undo this Action. The registrar can choose “Exclude” from the Action column: this changes the patient's Status to “Exclude Approved”. The patient record is removed from the list shown above. When this is chosen, an “Undo” button appears on the form that allows the registrar to undo that Action. The registrar can also click on Last Name field to review more details about the case before setting the Status (see Patient Review section below). This decision is made after the registrar reviews the systems recommendations in the Patient Review section of the system described below.

The system maintains the following statuses, and of course, others are within the scope of the present invention.
Pending—patient data has been received, but the data is not yet ready to process.
Exclude—patient data does not need to be abstracted because they do not meet the criteria of the destination registry.
Exclude Approved—the Exclude status above has been reviewed and approved by the registrar.
Include—the patient data has been processed for inclusion and meets the requirements for inclusion but needs registrar review for registry abstraction.
Inclusion Confirmed—the registrar confirmed the above Include status.
Processing—the system is currently creating registry records from the patient data for the particular patient.
Processing Edits—the system is re-processing patient data based on recent manual edits made by the registrar.
Ready for Registrar Review—the system has applied the registry rules are the registry records are ready for registrar review.
Sent—the patient record has been exported and sent to the registry.

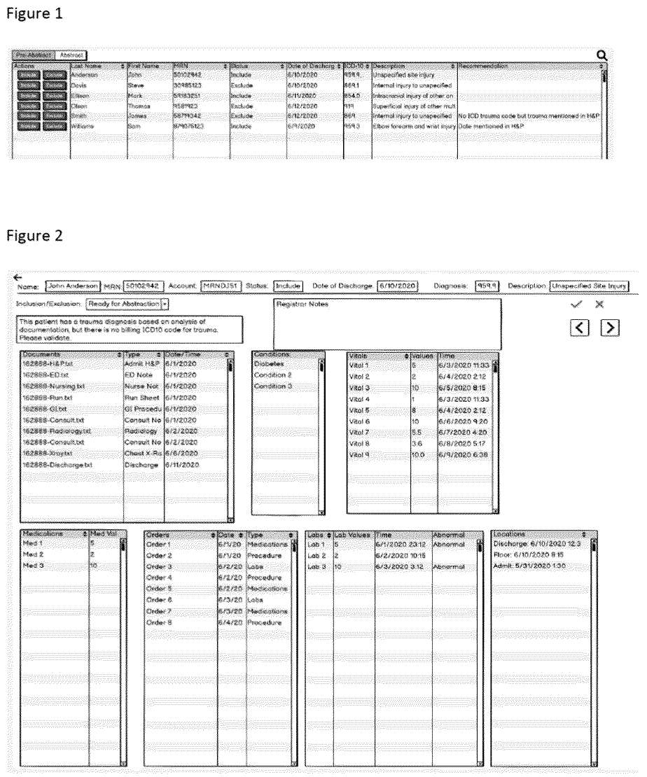

A further function of the system is the Patient Review process that allows the registrar to review all of the relevant clinical data for a patient and make the above status decisions and review the system recommendations, including determining whether a patient should be included or excluded from the registry, and deciding appropriate answers for a specific registry data field issues/questions presented by the system. The Patient Review screen is shown in FIG. 2, which is comprised of the patient data for one of the patients (John Anderson) shown in the Pre-Abstract Patient List shown above.

The Patient Review screen displays the following information:
Patient information in the header, including Name, MRN, Account, Status, Date of Discharge, Diagnosis, and Description.
The system's recommended status, either for inclusion/exclusion or for the specific data field.
Verbiage that explains the rationale for the system's abstract recommendation. Each recommendation is connected to information from the source systems (ex. EHR, billing system, EMS system) via hyperlinks in the text. Clicking on the hyperlinked words displays a list of the documents that are associated with the hyperlinked terms. Next to each document, the relevant sentence is displayed. If the registrar clicks on a document or the sentence next to it, the source document opens with the text highlighted for the registrar to review in further detail.
Registrar Notes field that allows the registrar to enter any comments. The Registrar Notes field and the recommendation field are the only two editable fields on the Patient Review screen.
Tables with the following clinical information are also displayed:
   Clinical Documents which are hyperlinked and, when clicked, bring up a separate form that displays the document.
   Medical Conditions
   Vitals
   Labs
   Medications
   Orders
   Locations
   These tables are sortable and searchable
Arrows with the following functionality:
   When using the form for inclusion/exclusion status determination/review, the arrows move to the next (or previous) patient on the Pre-Abstract Patient List.
   When reviewing a specific data field, the arrows move to the next (or previous) data field.
When reviewing a specific data field, double arrows allow the registrar to move to the next (or previous) data field with a red exclamation point. Fields with a red exclamation point are ones that the system has flagged for required registrar review and disposition.
Green check and red X buttons:
   Clicking the green check accepts whatever changes have been made to the system's recommendations and the Registrar Notes field. If the data field originally had a red exclamation point, that graphic is changed to a green check mark to indicate that registrar review is complete for that field.
   Clicking the red X cancels all changes the registrar made in the form.
   When either button is clicked, the registrar is returned to the relevant Patient List form.
Clicking the arrow in the upper left returns the registrar to the relevant Patient List form.

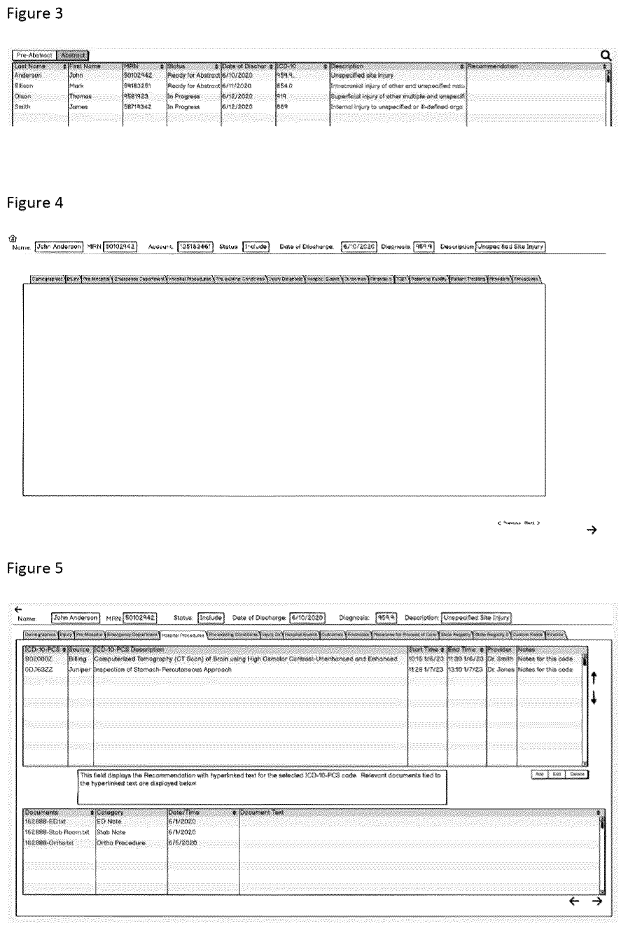

Next, the Abstract Patent List system functionality is described. The screen shown in FIG. 3 is displayed, and is related to the screen shown above for the Pre-Abstract step (except the Abstract” tab is selected). Once a patient's Status has been reviewed and approved by the registrar, the patients approved for abstractions appear on the Abstract Patient List and their cases are ready to be abstracted to the applicable registry. The registrar can access the Abstract Patient List by logging into the system and clicking the Abstract button at the top of the screen shown below.

The Abstract Patient List contains the following:
Table with these columns: Last Name, First Name, MRN, Status, Date of Discharge, ICD-10, Description, and Recommendation.
Each Last Name value is hyperlinked. Clicking on a hyperlink opens the Abstract Detail screen (see below).
The table displays the patients status (described above):
Search field: The search field can be used to search for any patient in the system. Searches can be done on Last Name, First Name, and Medical Record Number. When a search is performed, an “X” appears in the search field. The registrar can click on the “X” to return to the previous Patient List.
Column headers can be clicked to sort the data in the grid.
The ICD-10 and Recommendation columns may contain more text than what can be presented in the grid. When that occurs, the system displays an ellipsis in those cells and the registrar can hover the cursor over the relevant cell to display the entire text in a balloon.

All of the abstraction work is performed within the Abstract Detail screen shown in FIG. 4, where the initial screen appears as follows (for one of the patients shown in the Abstract Patient List shown above).

The screen is patient specific, and the header is the same as shown on the Patient Review screen. Each main section of the Abstract Detail screen appears as tabs below the header, and each tab can be selected to display the data under each section for the particular patient. The tabs that appear are configurable and may vary from site to site. The choices include the following: Demographics, Injury, Pre-Hospital, Emergency Department, Hospital Procedures, Pre-existing Conditions, Injury Diagnosis, Hospital Events, Outcomes Financials, Trauma Quality Improvement Program (TQIP) Referring Facility, Patient Tracking, Providers, Procedures, Diagnoses, QA, and Finalize.

Each tab section displays numerous fields of data taken from the patient's EHR record and placed into the appropriate data field specific to the applicable registry (for example the National Trauma Data Bank), or state-specific data dictionaries, or site-specific fields. Each tab section shares the following features:
The header for each section includes Name, MRN, Account, Status, Date of Discharge, Diagnosis, and Description.
A home button in the header takes the registrar back to the Abstract Patient List.
Next button and previous button in the lower right of each tabbed page. Clicking these buttons moves the registrar from one section to the next.
The Finalize section contains a “Submit” button. When clicked, the system checks to see if any fields still need to be populated or reviewed. If so, those fields are displayed for the registrar to address. The system also runs all Associated Edits and presents any issues with those edits. Once that process is complete, the patient's status is set to Final and the registrar is returned to the Abstract Patient List.

Each field within each section has the following labels:
Green check mark: the system has a high degree of confidence that the recommended answer for the particular field is correct.
Red exclamation point: fields that require registrar review.

The registrar is able to edit the information in either of these types of fields. To review more detail about the data field, the registrar can click the red exclamation point or the green check mark, which opens the Patient Review screen. The specific field that was clicked on is displayed, along with all other data fields, tables and functionality denoted in the Patient Review screen earlier in this document.

Once the registrar has completed their abstraction for a particular patient, they can proceed to the Final section and finalize the case.

An additional feature of the system is the Hospital Procedures functionality. In this section, the registrar can review all ICD-10-PCS codes (procedural coding system) as shown in the FIG. 5.

The above Hospital Procedures screen contains the following:
Table that lists the ICD-10-PCS Code, Source (billing system or Juniper), ICD-10-PCS Description, Start Time, End Time, Provider and Notes.
Up/down arrows to the right of the table, allow for re-ordering of the codes.
Window at the bottom of the screen that shows relevant supporting information for the procedure code selected in the table.
When a code is selected, the abstractor text appears in the window. As in the Patient Review screen, some words may be hyperlinked in the text. Clicking on hyperlinked words displays a list of the documents that are associated with the hyperlinked terms. Next to each document, the relevant sentence is displayed. If the registrar clicks on a document or the sentence next to it, the document opens with the text highlighted.
Codes can be added, edited, or deleted.

One of the functions performed by the system is Injury Diagnosis. In this section, the registrar can review all ICD-10-CM codes.

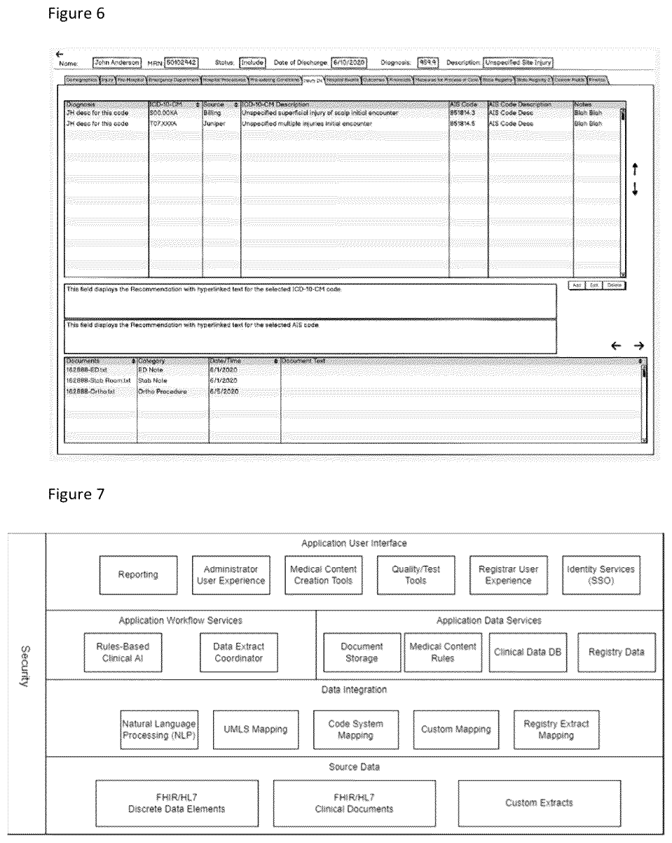

The Injury Diagnosis screen shown in FIG. 6 contains the following:
Table that lists the Diagnosis (this is a description created by Juniper), ICD-10-CM Code, Source (Billing or Juniper), ICD-10-CM Description, AIS Code, AIS Code Description and Notes.
Up/down arrows to the right of the table, allow for re-ordering of the codes.
Window at the bottom of the screen that shows relevant data for the diagnosis code selected in the table.
When a code is selected, the abstractor text is displayed in the window. As in the Patient Review form, some words may be hyperlinked in the text. Clicking on hyperlinked words displays a list of the documents that are associated with the hyperlinked terms. Next to each document, the relevant sentence is displayed. If the registrar clicks on a document or the sentence next to it, the document opens with the text highlighted.
Codes can be added, edited, or deleted.

The present invention relies on an NLP processing tool. NLP refers to the branch of computer science—and more specifically, the branch of artificial intelligence or AI—concerned with giving computers the ability to understand text and spoken words in much the same way human beings can. NLP combines computational linguistics—rule-based modeling of human language—with statistical, machine learning, and deep learning models. Together, these technologies enable computers to process human language in the form of text or voice data and to ‘understand’ its full meaning and context, complete with the speaker or writer's intent and sentiment. The present invention utilizes a commercially available NLP tool called by BioMedICUS provided by the University of Minnesota, which is a system for large-scale text analysis and processing of biomedical and clinical reports including EHR data; however, the invention is not limited to this particular tool.

The present invention relies on a complex rules engine to apply rules to the discrete data elements and NLP output to complete the abstraction process and provide a set of intelligent recommendations to the registrar that greatly simplifies the abstraction process. The rules are specifically tailored to the data that is processed and the data that the registry expects. The rules are functionally structured, such that sets of rules are applied to the data based on the broad nature of the data (for example, EHR, billing), and/or narrowly tailored to analysis of individual registry fields. Rules fall within the following categories:

| Rule Category | Description |
|---|---|
| Inclusion | These rules determine whether a patient meets |
| | the criteria to be included in registry submissions. |
| Data Derivation | These rules derive new, more complex data concepts |
| | through evaluation of simpler existing discrete and |
| | NLP data elements. This includes, for example, cases |
| | within NLP where a concept is derived from two |
| | NLP-identified data concepts appearing within the |
| | same sentence or paragraph. |
| Registry | These rules create registry-specific selections for |
| | submission based on the discrete, NLP, and complex/ |
| | derived data elements. |
| Associated Edit | These rules identify registry selection anomalies |
| | that violate the registry submission specification. |
| | This includes, for example, cases where the answer |
| | for one registry item is considered invalid based |
| | on the answer for another registry item. |

In this manner, the present invention automates the abstraction process by translating between the EHR data and other patient data, conforming it to the structured format/language that the registry requires, which requires interpreting and understanding the meaning of the data. This is a task that heretofore had to be done manually by the registrar. The system has specialized rules-based functions that can interpret the data collectively, and at the data field level, make recommendations for abstractions and provide its rationale for the recommendation—all of which the registrar can then review, revise, or accept; and the system then transfers the data to the applicable registry.

The present invention is carried out through software and other programing constructs operating on one or more computer systems, including in a network environment or via the Internet. In particular, the overall software system consists of the following functional and/or structural components: Source Data, Data Integration Services, Application Data Services, Application Workflow Services, and an Application User Interface. The software subsystems at the highest level are based on a layered architecture design as shown in FIG. 7.

The layered view of the system above divides the software layers, with multiple units in each layer. Each layer represents a virtual machine. Furthermore, there are constraints on the relationship among the virtual machines where upper layers are only allowed to communicate with adjacent layers. The exception to the relationship constraints are virtual machines or layers that are adjacent to all layers of the architecture.

The following table describes each layer and its content:

| Layer | Description |
|---|---|
| Source Data | Includes adapters for patient data sources |
| | including: |
| | HL7 APIs (FHIR) and data streams. |
| | Clinical Documents |
| | Custom Data Extracts including file drops |
| | and custom APIs |
| Data Integration | Includes implementations for processing |
| | incoming patient data via: |
| | NLP processing with UMLS terminology mapping |
| | to Performance Improvement Decision Support |
| | (PIDS) concepts. |
| | Discrete patient data processing with code |
| | system and custom mapping to PIDS concepts. |
| | System data processing with mapping to |
| | external system (e.g. public registry) |
| | standard schemas. |
| Application | Includes implementations of application event |
| Workflow | processors and workflow coordination. See |
| | Application - Event/Workflow Services below. |
| Application Data | Includes storage (database, file, BLOB, table, |
| Services | cache) services to support adjacent application |
| | layers. |
| Application User | Includes implementations of UIs ranging from |
| Interfaces | customer-facing applications to UIs supporting |
| | PIDS content creation to quality/test tools |
| | and Identity management. |

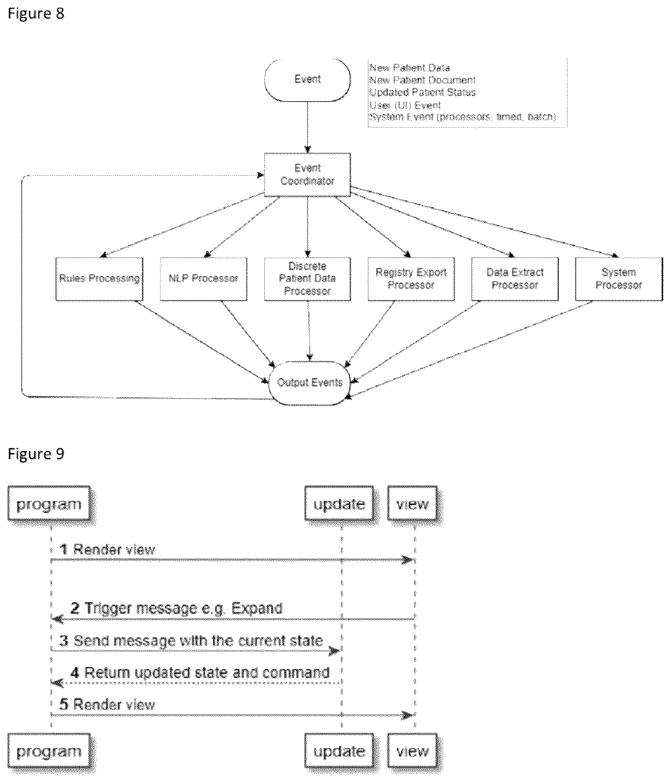

The system architecture, shown in FIG. 8, includes the following details, including the Workflows shown schematically below. In general, the system processing described herein happens in real time or near real time, such that at any time the system has processed all available information and is ready for the registrar to review the abstraction recommendations. The registrar does not have to pull data into the system for processing.

The system uses an event-driven architecture. An event-driven architecture consists of event producers that generate a stream of events in response to a condition that triggers system processing, and event consumers that listen for the events produced by the producers.

Events are delivered in near real time, so consumers can respond immediately to events as they occur. Producers are decoupled from consumers-a producer does not know (does not need to know) which consumers are listening. Consumers are also decoupled from each other, and every consumer sees all the events. This differs from a Competing Consumers pattern, where consumers periodically pull messages from a queue and a message is processed just once (assuming no errors).

In the logical diagram above, each type of consumer is shown as a single box. In practice, it is common to have multiple redundant instances of a consumer, to avoid having the consumer become a single point of failure in system. Multiple instances might also be necessary to handle the volume and frequency of events.

The following tables describe the high-level events and consumers driving the system.

| Event | Description |
|---|---|
| New Patient Data | New patient data is available from one of the |
| | many sources (FHIR, HL7v2, or Custom Jobs) in |
| | source data layer. |
| New Patient | A new patient document is available for processing |
| Document | from the source data layer. |
| Updated Patient | A system (e.g. rules processing) or user event has |
| Status | triggered a change in patient status in the system |
| | workflow. |
| User Event | The user has modified data or taken some other |
| | action on a patient that may trigger additional |
| | system processes. |
| System Events | Events that represent cross-cutting application |
| | concerns including timed events, failure events, |
| | or informational events for logging. |

| Processor | Description |
|---|---|
| Rules Processor | Handles events that require applying PIDS rule sets |
| | to new or updated patient data. |
| NLP Processor | Handles document events that require running a |
| | patient document through NLP processing to record |
| | new medical concepts. |
| Discrete Patient | Handles new or updating patient data by mapping the |
| Data Processor | data to concepts used in PIDS ruleset processing. |
| Registry Export | Handles system or user-generated requests to export |
| Processor | RAS data to external systems including reporting |
| | registries. |
| Data Extract | Handles user requests to export RAS data to external |
| Processor | systems including reporting systems. |

User interfaces are developed using the commercially available tools such as the Model-View-Update pattern via the F #Elmish library within the Bolero F #WebAssembly application framework (see FIG. 9).

The goal of the architecture is to provide a solid user interface independent core to build the rest of the functionality around. Elm architecture operates using the following concepts, as they translate to Elmish:

| Concept | Description |
|---|---|
| Model | This is a snapshot of an application state, defined as |
| | an immutable data structure. |
| Message | This an event representing a change (delta) in the state of |
| | the application, defined as a discriminated union. |
| Command | This is a carrier of instructions, that when evaluated may |
| | produce one or more messages. |
| Init | This is a pure function that produces the initial state of |
| | the application and, optionally, commands to process. |
| Update | This is a pure function that produces a new state of |
| | the application given the previous state and, |
| | optionally, new commands to process. |
| View | This is a pure function that produces a new UI |
| | layout/content given the current state, defined as an F# |
| | function that uses a renderer (such as React) to |
| | declaratively build a UI. |
| Program | This is an opaque data structure that combines all of the |
| | above plus a setState function to produce a view from the |
| | model. |

Unless otherwise defined, all technical and scientific terms used herein have the same meaning as commonly understood by one of ordinary skill in the art to which this invention belongs. Although methods and materials similar to or equivalent to those described herein can be used in the practice or testing of the present invention, suitable methods, and materials are described below. All publications, patent applications, patents, and other references mentioned herein are incorporated by reference in their entirety to the extent allowed by applicable law and regulations. In case of conflict, the present specification, including definitions, will control.

The present invention may be embodied in other specific forms without departing from the spirit or essential attributes thereof, and it is therefore desired that the present embodiment be considered in all respects as illustrative and not restrictive, reference being made to the appended claims rather than to the foregoing description to indicate the scope of the invention. Those of ordinary skill in the art that have the disclosure before them will be able to make modifications and variations therein without departing from the scope of the invention.

1. A method for data abstraction carried out under the control of computer processing control, comprising:
accessing patient health record data from an EHR system;
processing and interpreting the data with a computer controlled natural language tool;
abstracting structured data elements from the data; and
transmitting the data to a data registry.

2. The method of claim 1 where the natural language tool extracts from the data medical terms and associated medical codes along with additional contextual information.

3. The method of claim 1 further comprising the step of applying optical character recognition technology to the data to create a text file for processing by the natural language tool.

4. The method of claim 1 further comprising a pre-abstraction step screening the data to determine what data is abstracted.

5. The method of claim 4 where a rules engine performs the pre-abstraction step resulting in a status assigned to the data to indicate whether the data is to be abstracted.

6. The method of claim 5 where the status determination is reviewed by a human.

7. The method of claim 5 further comprising the step of creating from the data a list of patients records ready for abstracting.

8. The method of claim 1 where the abstracting step requires structuring the data from the EHR system to align with the structure of the data registry.

9. The method of claim 1 where the data include medical coding data.

10. The method of claim 1 where the data include injury diagnosis data.

11. The method of claim 5 where the rules engine includes categories of rules for patient inclusion, data derivation, registry specific rules, and resolution of data anomalies.
